(19) United States
(12) Patent Application Publication
(10) Pub. No.: US 2011/0306889 A1
(43) Pub. Date: Dec. 15, 2011

(54) Pulse Condition Acquisition Apparatus And Method

(76) Inventors: Dan Di, Shanghai (CN); Zhe Pan, Shanghai (CN); Yue Pan, Shanghai (CN); Weichang Tang, Shanghai (CN)

(21) Appl. No.: 13/177,289

(22) Filed: Jul. 6, 2011

Related U.S. Application Data
(63) Continuation of application No. PCT/CN2010/000661, filed on May 10, 2010.

(30) Foreign Application Priority Data
Sep. 21, 2009 (CN) 200910057920.6

(51) Int. Cl. A61B 5/022; A61B 5/021

(52) U.S. Cl. 600/499; 600/485

(57) ABSTRACT

A device and method for collecting sphygmus information; it includes a sensor component, said sensor component incorporates a electric machine fixed in the casing, a screw fixed with said electric machine, a lifting structure setting outside said screw, and a sensor probe fixed in the base of said lifting structure, said electric machine drives said screw to revolve when rotating, further makes said lifting structure ascended or descended relatively to said screw. The pulse signal collection can be automatically achieved, and limited protecting structure is employed in the collecting process.

### CROSS-REFERENCE TO RELATED APPLICATIONS

The present application is a continuation of pending International patent application PCT/CN2010/000661 filed on May 10, 2010 which designates the United States and claims priority from Chinese patent application 200910057920.6 filed on Sep. 21, 2009, the content of which is incorporated herein by reference.

### FIELD OF THE INVENTION

The present invention relates to a TCM diagnostic device and in particular to a device and method for collecting sphygmus information. The invention also relates to a sphygmus collecting method.

### BACKGROUND OF THE INVENTION

The diagnostic methods the practitioner of Chinese medicine frequently used include inspection, auscultation and olfaction, interrogation and palpation, among which palpation is one of the important diagnostic methods. The practitioner of Chinese medicine feeling the pulse with fingers is to detect pulse information through tactile sensation, pressure sensation, palmesthesia receptors under different pressures (that is floating pressure, medium pressure, heavy pressure) the figure tips bringing to bear on the artery pulse, which includes the information of the number of times the heart beats per minute, rhythm, drifting, strength-thickness, rigid-flexible, fluent, abstruse and so on. Therefore, the TCM sphygmus information should be interpreted as the collection of the pressure of feeling the pulse and the pulsation power of pulse wave.

The existing pulse detecting devices are usually using the manual pressure mode, for example, the description for the Chinese utility model CN2420975Y (announced on Feb. 28, 2001) disclosed an intelligent TCM pulse detecting device (Please refer to FIG. 1). Defects in the above-mentioned device is that it cannot find the optimal measuring position accurately, and the information collecting position cannot be repeatedly achieved.

With the development of technology, people began to use automatic pressure measuring mode, but the predetermined pressurizing mode is usually controlled by procedure or computer, and the limit protecting structures are not employed in hardware, so the sensor probe will press people's pulse all the time when program error occurs, which will cause harm to people when errors occur in the program and the sensor probe continuously presses the pulse of the patient.

### SUMMARY OF THE INVENTION

The technical problems the present invention solves is to provide a kind of device for collecting sphygmus information, by which automatically collection of sphygmus information can be achieved, and limit protecting structure is employed in the collection process. Accordingly, the invention also provides a method for collecting sphygmus information.

To solve the above technical problems, the present invention provides a device for collecting sphygmus information, including a sensor component, said sensor component comprises an electric machine fixed in the casing, a screw fixed with said electric machine, a lifting structure positioned outside said screw, and a sensor probe fixed in the base of said lifting structure, said electric machine drives said screw to revolve when rotating, further makes said lifting structure ascended or descended relatively to said screw.

Based upon the above-mentioned device, the invention provides a kind of method for collecting sphygmus information, which includes the steps as follows:

(1) the rotating electric machine drives the lifting structure to ascend to the initial position.

(2) the electric machine drives the lifting structure to descend a preset distance, then determines whether the lifting structure reaches the limiting position; if it does, the measurement will stop, and if it does not, the sensor probe will collect the pressure signal.

(3) repeat step (2), and determine whether the signal collected is stronger than that collected last time; if it is not, the measurement will stop.

The invention with above-mentioned structure has the following advantages:

1. collecting process for pulse signal is automatically achieved by electric machine, the number of turns the electric machine revolving determines the stroke the probe ascends or descends, which ensures the accuracy and repeatability of the collecting position.

2. (the limited position of the lifting structure stroke is set up by the optocoupler, which avoids the harm to people when procedure error occurs and the sensor probe continuously presses the pulse of the patient).

use optocoupler to set up the limiting travel position of the lifting structure to avoid doing harm to people when procedure error occurred.

### BRIEF DESCRIPTION OF THE DRAWINGS

The invention will now be further described in details as follows

FIG. 1 is an existing device for collecting sphygmus information.

FIG. 2 is the schematic diagram of the device for collecting sphygmus information in active condition, according to the present invention.

FIG. 3 is the schematic diagram of the positioning component, according to the present invention.

FIG. 4 is the schematic diagram of the sensor component, according to the present invention.

FIG. 5 is the schematic diagram of the internal construction of the sensor component, according to the present invention.

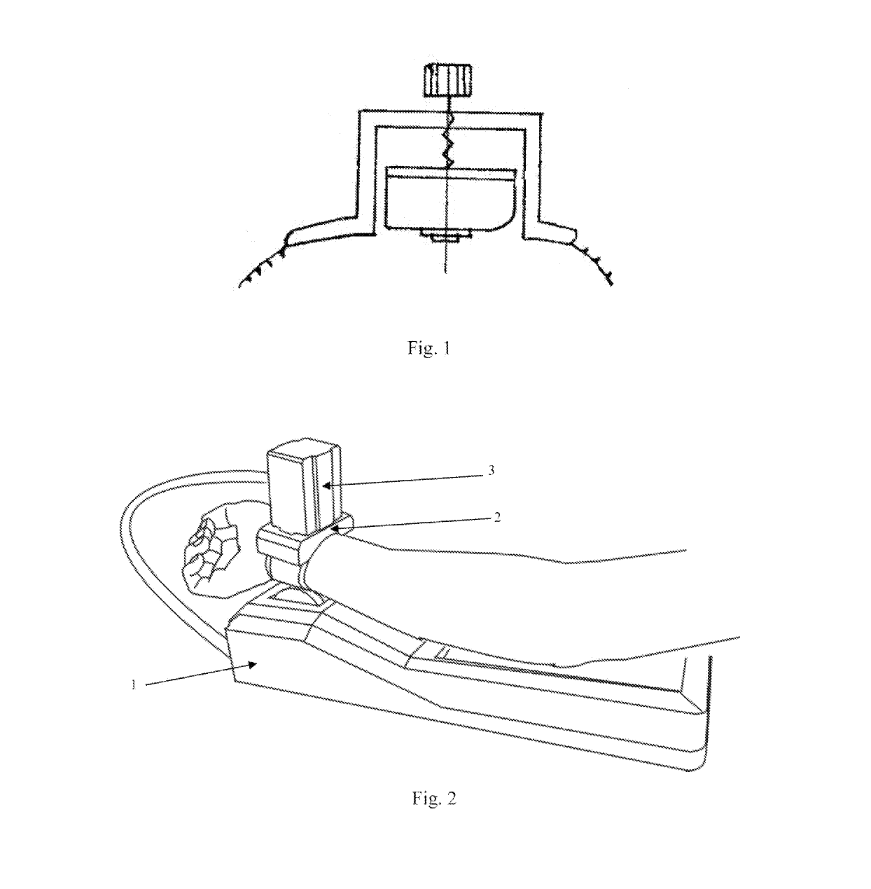

The marks in the figures are: 1. base; 2. positioning component; 21. positioning window; 22. positioning mark; 23. slot; 3. sensor component; 31. electric machine; 32. optocoupler; 33. screw; 34. lifting structure; 41. labe; 342. guide; 35. sensor probe; 36. collecting window.

### DETAILED DESCRIPTION OF THE INVENTION

As shown in FIG. 2, it is one embodiment of the device for collecting sphygmus information in the present invention; the device comprises a base 1, a positioning component 2 and a sensor component 3. when using, the positioning component should be fixed around the wrist first and then the sensor component 3 is installed in the positioning component for detecting.

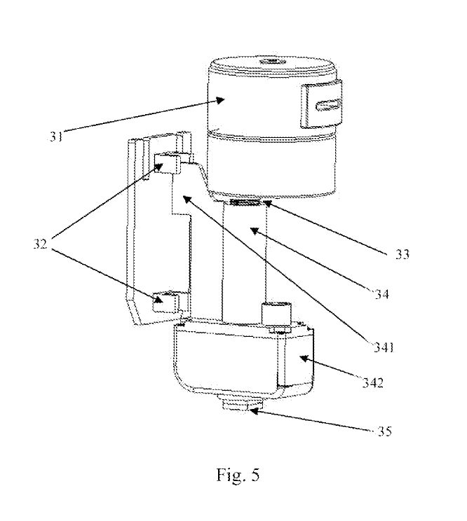

One embodiment of the sensor component in the present invention is shown in FIG. 4 and FIG. 5. FIG. 4 is the schematic diagram of the external construction; FIG. 5 is the schematic diagram of the internal construction. The sensor component 3 in the present invention includes an electric machine 31 fixed in the casing, a screw 33 fixed with said electric machine 31, a lifting structure 34 positioned outside said screw 33, and a sensor probe 35 fixed in the base of said lifting structure 34. The lifting structure 34 and the screw 33 are interconnected by screw thread. There is a guide 342 in the base of the lifting structure 34, and the guiding component 342 is stuck in the non-circular capture window (Such as square or rectangular) placed in the base of the sensor component 3; when making clockwise rotation or anti-clockwise rotation, the electric machine will drive the screw 33 to revolve, as the guide 342 and capture window are designed to be non-circular, the guide 342 is stuck in the capture window and cannot revolve with the screw 33, which further makes the lifting structure 34 ascended or descended relatively to the screw 33.

Preferably, there is a labe 341 in the lifting structure 34, and the sensor component comprises at least one optocoupler 32 fixed in the sidewall of the casing; when the lifting structure 34 ascending or descending, the labe will pass through the optocoupler 32.

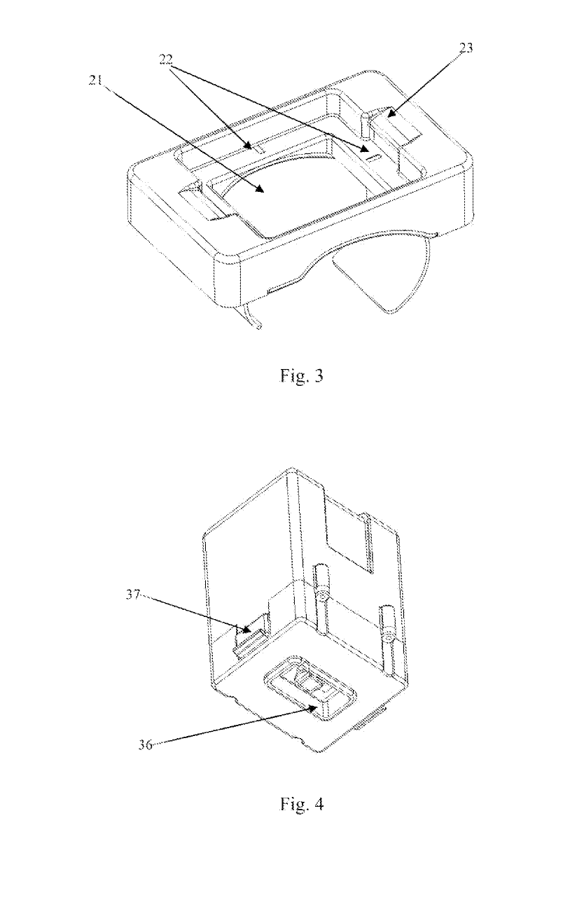

As shown in FIG. 3, it is one embodiment of the positioning component, there is a positioning window 21 in the positioning component 2 and a collecting window 36 at the bottom of the sensor component 3 (please refer to FIG. 4), wherein the sensor probe can pass through the collecting window 36 and positioning window 21 and press on the wrist to collect pulse signal. A snap-fit 37 is placed in the casing of the sensor component 3, and in the outer frame of the sensor component 3 there places a slot 23; the snap-fit 37 is stuck in the slot 23 when installed.

As shown in FIG. 5, the two optocouplers 32 of upper and lower [symbol] can be placed, and each positioned at the labe's position respectively when the sensor probe is working at the initial position and the limiting position.

Around the positioning window 21 there are positioning marks 22 for directing the pulse's position. The positioning marks 22 can be set in the centre of the four sides of the positioning window, and correspondingly the sensor probe is positioned in the centre of the collecting window.

The method for collecting sphygmus information in the present invention includes the steps as follows:

(1) the electric machine drives the lifting structure to ascend to the initial position. The method to determine whether the lifting structure reaches the limited position is: to determine whether the labe of the lifting structure reaches the position of the first optocoupler.

(2) the rotating electric machine drives the lifting structure to descend of a preset distance, and determine whether the lifting structure reaches the limiting position, if it does, the measurement will stop, if it does not, the sensor probe will collect pressure signal. The method for determining whether the lifting structure reaches the limiting position is that determine whether the labe of the lifting structure reaches the position of the second optocoupler. Preferably, the stroke of the sensor probe working in the initial position and in the limiting position ranges between 0 mm to 25 mm, and people are likely to be hurt without setting the limited position.

(3) repeat step (2) determines whether the signal collected is stronger than that collected in the previous time, if it is not, the measurement will stop. In the process of collecting pulse signal, the press signal collected is determined by the depth the sensor probe pressurize on the wrist, wherein the signal the sensor received will be extremely weak while pressing too lightly, and while pressing too heavily, the pulsation of the radial artery will be affected, and the signal the sensor received will be attenuated. So in the collecting process proper pressure will collect the strongest signal. In the process from the initial position to the limited position, signal is gradually enhancing, and when signal collected in a certain position is weaker than the previous one, it indicates that the pressure pressurized is the optimum, then the collection can be terminated to save measurement time.

What is claimed is:

1. A kind of device for collecting sphygmus information, characterized in that, it includes a sensor component, said sensor component incorporates a electric machine fixed in the casing, a screw fixed with said electric machine, a lifting structure setting outside said screw, and a sensor probe fixed in the base of said lifting structure, said electric machine drives said screw to revolve when rotating, further makes said lifting structure ascended or descended relatively to said screw.

2. The device for collecting sphygmus information as set forth in claim 1, characterized in that, said lifting structure and said screw are interconnected by screw thread, there is a guide in the base of said lifting structure, the guide is stuck in a non-circular collecting window positioned in the base of said sensor component.

3. The device for collecting sphygmus information as set forth in claim 1, characterized in that, said lifting structure has a labe, said sensor component also incorporates at least one optocoupler fixed in the sidewall of the casing, when said lifting structure is ascending or descending, said labe passes through said optocoupler.

4. The device for collecting sphygmus information as set forth in claim 3, characterized in that, said optocoupler number is two, both respectively positioned in the labe's positions when said sensor probe working respectively in the initial position and the limiting position.

5. The device for collecting sphygmus information as set forth in claim 1, characterized in that, said acquisition device for sphygmus information also incorporates a positioning component in connection with said sensor component, said positioning component has a positioning window, the base of said sensor component has a collecting window, said sensor probe passes through said collecting window and press on the wrist to collect pulse signal.

6. The device for collecting sphygmus information as set forth in claim 5, characterized in that, in the casing of said sensor component there is a snap-fit, in the outer frame there is a slot, when installing, said snap-fit is fitted into said slot.

7. The device for collecting sphygmus information as set forth in claim 5, characterized in that, around said positioning window there are positioning marks for directing the position of pulse.

8. The device for collecting sphygmus information as set forth in claim 5, characterized in that, said positioning marks are positioned in the center of the four sides of said positioning window, said sensor probe is positioned in the center of said collecting window.

9. A method for collecting sphygmus information, including the steps of:
(1) the electric machine drives the lifting structure to ascend to the initial position;
(2) the electric machine drives the lifting structure to descend of a preset distance, determines whether the lifting structure reaches a limiting position, if it does, the measurement will stop, if it does not, the sensor probe will collect a pressure signal;
(3) repeat step (2), determine whether the signal collected is stronger than that collected in step (2), if it is not, the measurement will stop.

10. The device for collecting sphygmus information as set forth in claim 9, characterized in that,
the method for determining whether the lifting structure reaches the limiting position in said step (1) is that determine whether a labe of the lifting structure reaches the position of the first optocoupler.
the method for determining whether the lifting structure reaches the limiting position in said step (2) is that determine whether the labe of the lifting structure reaches the position of the second optocoupler.

11. The device for collecting sphygmus information as set forth in claim 9, characterized in that, the stroke of the sensor probe working at the initial position and the limiting position ranges between 0 mm to 25 mm.
